(19) United States
(12) Patent Application Publication
(10) Pub. No.: US 2025/0087300 A1
(43) Pub. Date: Mar. 13, 2025

(54) SYSTEMS AND METHODS FOR SELECTING RECOMMENDED CROSSES WITH INCREASED AN PROBABILITY OF MEETING PLANT-BASED PRODUCT SPECIFICATIONS

(71) Applicant: Benson Hill, Inc., St. Louis, MO (US)

(72) Inventors: Bryan Goggin, St. Louis, MO (US); Avjinder Singh Kaler, St. Louis, MO (US); Craig Rolling, St. Louis, MO (US); Paul Skroch, St. Louis, MO (US)

(21) Appl. No.: 18/725,456

(22) PCT Filed: Dec. 30, 2022
(86) PCT No.: PCT/US22/54398

§ 371 Date: Jun. 28, 2024

Related U.S. Application Data
(60) Provisional application No. 63/295,663, filed on Dec. 31, 2021.
(60) Provisional application No. 63/295,796, filed on Dec. 31, 2021.
(60) Provisional application No. 63/295,822, filed on Dec. 31, 2021.
(60) Provisional application No. 63/326,729, filed on Apr. 1, 2022.
(60) Provisional application No. 63/326,745, filed on Apr. 1, 2022.

(51) Int. Cl. G16B 20/00; G06F 30/27; G16B 40/00

(52) U.S. Cl. CPC G16B 20/00; G06F 30/27; G16B 40/00

(57) ABSTRACT

A computer-based method for selecting recommended crosses from a population of plants with an increased probability of meeting a plant-based product specification, comprising: (a) collecting plant data for the plant population including at least labelled parentage information including genetic and phenotype information; (b) training a machine learning model mapping phenotypes to genotype based on the collected data; (c) extracting a target list including one or more phenotypes needed to meet the product specification; (d) simulating pairwise combinations of one or more available parents using rapid recombination simulation; (e) applying the phenotype-to-genotype mapping to predict phenotypes for each simulated combination; (f) selecting the simulated combinations that meets phonetic criteria on target list; (g) simulating selfed combinations of each selected simulated combination using rapid recombination simulation; (h) repeating (e) through (g) until F3 generation is simulated; and (i) creating a predictive crossing list of simulated F3 progeny that meets the product specification.

### CROSS REFERENCE TO RELATED APPLICATIONS

This application claims the benefit and priority of U.S. Provisional Patent Application Ser. No. 63/295,663 filed Dec. 31, 2021 entitled “Systems And Methods For Training A Machine-Learning Model And Subsequently Applying That Machine Learning Model To Recommend Best Crosses Within An Existing Germplasm For Plant Breeding,” U.S. Provisional Patent Application Ser. No. 63/295,796 filed Dec. 31, 2021 entitled “An Optimized Set of Genomic Markers for Use in Soybean Breeding Programs With and Without Machine Learning Modeling,” U.S. Provisional Patent Application Ser. No. 63/295,822 filed Dec. 31, 2021 entitled “Systems and Methods for Recommending Cross Breeding,” U.S. Provisional Patent Application Ser. No. 63/326,729 filed Apr. 1, 2022 entitled “An Optimized Set of Genomic Markers for Use in Soybean Breeding Programs With and Without Machine Learning Modeling,” U.S. Provisional Patent Application Ser. No. 63/326,745 filed Apr. 1, 2022 entitled “Systems And Methods For Accelerate Speed To Market For Improved Plant-Based Products,” the disclosures of which are hereby incorporated by references in their entirety.

### BACKGROUND

Genomics has been used for decades to develop crops for our food system, but most agricultural companies have focused almost exclusively on increasing the yield of a few crops, resulting in commodity ingredients and a food system based on the quantity of calories available. While focus on quantity is important, that focus resulted in lower nutrient density and changed flavors. Minimal diversity in ingredient options also led food manufacturers to add costly water- and energy-intensive processing steps, and additives like sugar and salt to make up for attributes that were muted in crops over time.

However, consumers are now demanding food choices with simpler ingredients that benefit their health and the health of our planet. Food- and diet-related health issues, including obesity and diabetes, are some of the most widespread health issues today and continue to increase. More than 65% of American adults are either overweight or have obesity and, according to the Centers for Disease Control and Prevention, approximately 90% of Americans do not eat the recommended daily amount of fruits and vegetables. Americans spend more on diet-related illnesses than on food itself.

Moreover, the current food system has a substantial environmental impact on the planet. According to an April 2020 report entitled “Agriculture and climate change” prepared by McKinsey & Company, twenty-seven percent of total greenhouse gas emissions (e.g., methane and nitrous oxide) are caused by agriculture, with cattle and dairy cows alone contributing eight gigatons of carbon dioxide equivalent (GtCO2e) emissions in 2019. (Accessed Dec. 22, 2021 at https://www.mckinsey.com/˜/media/mckinsey/industries/agriculture/our %20insights/reducing %20agriculture %20emissions %20through %20improved %20farming %20practices/agriculture-and-climate-change.pdf.)

At the same time, demand for plant-based solutions to feed the world and improve the environment is growing. Consumers are open to changing their eating habits to minimize further harm to the environment. Moreover, people are actively trying to incorporate more plant based foods into their diets, especially protein alternatives found in the meat and dairy grocery store sections. NielsenIQ Sep. 9, 2021 article entitled “Growing demand for plant-based proteins” (Accessed Dec. 22, 2021 at https://nielseniq.com/global/en/insights/analysis/2021/examining-shopper-trends-in-plant-based-proteins-accelerating-growth-across-mainstream-channels/).

The largest commercial source of plant protein today is the soybean plant. Other plant-based protein crops include chickpeas, edamame, lentils, peanuts, and peas.

### Soybeans: Generally

Soybeans are believed to have originated on the Asian Continent (glycine soja) where it is believed they were also first domesticated in China (glycine max). Abstract, Hymowitz and Newell, Taxonomy of the genus Glycine, domestication and uses of soybeans. Econ Bot 35, 272-288 (1981). Soybeans are a common field crop with the largest producing countries including the United States, Brazil, Argentina, China, India, Paraguay, and Canada. In the United States in 2020 soybeans were primarily produced in the Western Corn Belt (48.7%), Eastern Corn Belt (32.7%), and the Midsouth (11.9%) with Illinois and Iowa being the largest producing states. Naeve and Miller-Garvin, United States Soybean Quality 2020 Annual Report (Published by the University of Minnesota with the support of the United Soybean Board).

Soybean plants produce seed-bearing pods, each generally having 2-4 seeds. The seeds are harvested and processed either for future planting (i.e., to produce additional soybean plants) or processed into dozens of products (e.g., bean curd, feed for livestock, flour, meal, oil (cooking and industrial)). Soy flours includes flour concentrates and isolates, which are the primary protein products of soy.

Soybean seeds are usually planted in rows in soil. According to the 2012 Illinois Soybean Production Guide, soybeans require 55-60° F. soil temperature, an air temperature of at least 68° F., about 25 inches of water, sufficient nitrogen and five months from germination to harvest.

The radical (or root) is the first structure to emerge from a germinating soybean seed. The hypocotyl is the seedling structure that emerges from the soil surface. As the hypocotyl emerges it forms a crook as it pulls the cotyledons (i.e., the plant's first leaves) from the soil. Then, the cotyledons can unfold and begin the process of photosynthesis. Once the cotyledons have emerged from the soil surface the plant is said to be at the VE stage of vegetative development. The VC (cotyledon) development stage occurs once two unifoliate (or single blade) leaves emerge from opposite sides of the main stem and no longer touch the cotyledons. The V1 (vegetative) development stage occurs once the unifoliate leaves are fully expanded establishing the first node. V2 is defined as the stage wherein a second node (with a trifoliate leaf (i.e. three or four leaflets per leaf)) has formed above the unifoliate node. With the formation of each subsequent node “n” (n=3, 4 . . . ) with fully developed leaves the plant is referred to as being in the Vn development stage. Soybean farmers typically refer to the leaves and stems as the canopy.

The length of time for these vegetative and reproductive stages (discussed below) depends on the plant's maturity group (“MG” (i.e., the length of time from planting to physical maturity), the soil and air temperatures, and day length. Soybeans are short-day plants (i.e., the soybean plant is triggered to flower as the day length decreases below some critical value, which differs among MGs). See, e.g., Purcell, Salmeron and Ashlock, “Chapter 2: Soybean Growth and Development” Arkansas Soybean Production Handbook (University of Arkansas Division of Agricultural Research & Extension, 2014 Update). Soybeans planted in Arkansas tend to be MG3 through MG6. Id. In Illinois, where soybeans may be grown in regions traditionally understood to be in MG2 through MG5, the 2012 Illinois Soybean Production Guide notes that MG 5 to MG 8 soybeans tend to be determinate (i.e., they cease vegetative growth when the main stem terminates in a cluster of mature pods) and MG 0 to MG 4.9 tend to be indeterminate (i.e. they develop leaves and flowers simultaneously after flowering begins).

Each soybean plant can produce a lot of flowers. The flowers are small and hidden underneath the leaves of the plant. The number of flowers produced depends upon the number of nodes on the main stem and branches with flower-bearing nodes. Not all flowers produce pods. For those flowers that do produce pods whether the resulting pod produces a full complement of seeds requires ample nitrogen, sugar, other nutrients, and favorable environmental conditions.

When a soybean plant begins to flower, it is referred to as being in its reproductive (R) growth stage. Soybeans are a normally self-pollinating crop, in fact, they have a perfect flower structure for self-pollination. Still, bees have been known to be attracted to soybean flowers and cross-pollinated plants. Where cross-pollination is desired breeders need to intervene to prevent self-pollination: the pistil of a soybean plant can become mature and the anthers can begin to shed pollen before the soybean flowers even bloom, breeders seeking to cross-pollinate need to be proactive.

Soybean plants have eight reproductive stages: R1 (beginning flowering/bloom (i.e., at least one flower)), R2 (full flowering/bloom (i.e., an open flower at one of the two uppermost nodes)), R3 (beginning pod (i.e., a pod measuring 3/16 inch at one of the four uppermost nodes)), R4 (full pod (i.e., a pod measuring ¾ inch at one of the four uppermost nodes)), R5 (beginning seed (i.e., a seed measuring ⅛ inch long in the pod at one of the four uppermost nodes)), R6 (full seed (i.e., a pod containing a green seed that fills the pod at one of the four uppermost nodes)), R7 (beginning maturity (i.e., one normal pod has reached mature pod color)), and R8 (full maturity (i.e., at least 95% of pods have reach full mature color)).

As the days get shorter and the temperatures get cooler, the leaves on soybean plants begin to turn yellow, they subsequently turn brown, fall off, and expose the matured pods of soybeans. The soybeans are now ready to be harvested using combines. The header on the front of the combine cuts and collects the soybean plants. The combine separates the soybeans from their pods and stems, and collects them into some container.

After harvesting the soybeans are processed. The soybeans are cleaned, heat dried, crushed and then flaked. Thereafter, the flake is further processed. The primary method for further processing is referred to as the extraction or solvent process, as it uses organic solvents (e.g. hexane) to recover the soybean oil and protein from the flake. Aside from its substantial use of solvents, this process consumes significant amounts of energy.

### Soybeans: Seed Varieties, Breeding, and Genetic Modification

Today, there are literally thousands of varieties of soybeans. These soybeans are the result of hundreds of years of selective breeding. Selective breeding is the process of selectively propagating plants with more desired traits (often called “phenotypes”) and eliminating plants with less desired phenotypes. Breeding generations are often designed F1, F2, etc, (wherein the “F” stands for “filial”). It may further involve crossing two plants to produce one or more new varieties.

Plant botanists have understood since the days of Gregor Mendel, that plants may exhibit dominant or recessive phenotypes/traits (e.g., seed shape, flower color, seed coat tint, pod shape, unripe pod color, flower location, and plant height). Through his experiments on pea plants, Mendel further taught that the genotype of a particular phenotype is not necessarily correlated because that phenotype may result from homozygous dominant, heterozygous, or homozygous recessive alleles. Where the phenotype is dominant, it will be exhibited by either of the first two zygosities. Whereas a recessive phenotype can only be exhibited by the third, homozygous recessive example.

Homozygous genotypes breed true from generation to generation, while heterozygous genotypes do not. Thus, after finding a desirable phenotype, plant breeders work to develop homozygosity in the population, and then release the resulting pure line as a new variety. For example, hybrid varieties are the result of crossing two homozygous, but unrelated pure lines of a species. The resulting F1 of the cross are all heterozygous. However, by F2 50% of the plants are either homozygous (dominant or recessive) and by F3 heterozygosity is reduced to 25%. Once a desired trait is found in homozygous plants, commercial quantities are produced by replanting the resulting seeds over several generations.

However, it takes time for each generation of plants to grow from seeds to adult plants and time to cross plants once they've produced reproductive organs. These and other practical constraints of biology are natural obstacles for traditional breeding programs and slow the advancement of potential commercial products through the phases of a traditional commercial product pipeline. Moreover, while the foregoing basic principles of plant genetics are relatively straightforward, the issue of creating a commercially-desirable variety is complicated by the fact that breeders cannot isolate a particular phenotype from other traits that might be present elsewhere in the plant's genome. Even in Mendel's pea experiments where he worked with only seven traits, each having at least two phenotypes (e.g., seed color: green or yellow) existing with the six other traits each of which also having multiple phenotypes the number of potential combinations explodes given all the ways the phenotypes of each trait can combine with the phenotypes of all the other traits. In the case of the soybean (i.e., Glycine max), its genome has approximately 1,100M base-pairs packaged into twenty chromosome pairs. Arumuganathan K, Earle E D. Nuclear DNA content of some important plant species. Plant molecular biology reporter. 1991 August; 9(3):208-18. Thus, there are an infinite number of potential genetic combinations within the soybean genome. As should be readily apparent, due to the sheer size of a plant's genome, the number of traits/phenotypes, and other practical constraints of biology, traditional plant breeding requires significant time to establish new phenotypes in a population.

Presently, breeders are using the subjective approach to make crosses, which are based on their experiences and phenotype data of the parents. Breeders make a large number crosses and most of those crosses do not result in targeted commercial product. Further, lot of resources are invested in testing of those crosses in the field. This increases the cost of product development and has less-reliable outputs.

Among the more desirable characteristics that have been selectively bred in soybeans are increased yield and increased tolerance to various potential environmental stressors (e.g., insects, drought). Unfortunately, according to the United States Soybean Quality 2020 Annual Report (conducted by Naeve and Miller-Garvin of the University of Minnesota), while soybean yields have significantly increased in the United States over the last thirty years, the amount of protein contained in those soybeans has substantially declined over the same time period.

### Using Machine Learning to Improve Agricultural Ingredients

While protein output in soybeans has been decreasing, the demand for plant-based protein has been growing. So much so that the demand will likely not be fully met using current breeding, genetic engineering, agronomic, and processing technologies. The current commodity food system can take on the order of six to ten years to improve crops with quality attributes, assuming the agricultural industry can even find the genetic synergy to create the right germplasm and then figure out how to best enable the desired breeding.

There are other plants that could benefit from improved characteristics.

Machine learning (and other forms of artificial intelligence) are already being used to improve certain outcomes in agriculture. One key to successful machine learning is identifying the right types of data to gather and then using that data to train the right type of model. Another key may include identifying the wrong, unnecessary, or cumbersome data the inclusion of which is either unhelpful in developing the model or unnecessarily slows down or other makes the training process unnecessarily expensive without sufficient improvement of the model.

### SUMMARY OF THE DISCLOSURE

The present disclosure is directed to systems and methods for selecting recommended crosses from a plurality of seeds in an existing germplasm with an increased probability of meeting a plant-based product specification. The method comprises: (a) collecting into a database, with a processor, plant data for the population of plants in the germplasm, such plant data comprising at least labelled parentage information that includes genetic and phenotype information; (b) training, with the processor, a machine learning model mapping phenotypes to genotype based on the data collected into the database; (c) extracting, via the processor, a target selection list including one or more phenotypes needed to meet the plant-based product specification; (d) simulating, via the processor, pairwise combinations of one or more available parents using rapid recombination simulation; (e) applying, via the processor, the phenotype-to-genotype mapping to predict one or more phenotypes for each simulated pairwise combination; (f) selecting, via the processor, the simulated pairwise combinations that at least meets phonetic criteria on the target selection list; (g) simulating, via the processor, selfed combinations of each selected simulated pairwise combinations using rapid recombination simulation; (h) repeating (e) through (g) until an F3 generation has been simulated; and (i) creating a predictive crossing list of simulated F3 progeny that meets the product specification.

The method may further comprise ranking potential crosses on the predictive crossing list to promote one or more goals selected from the group comprising: (a) maximizing one or more desired phenotypic results; (b) ensuring diversification across maturity subgroups; (c) ensuring genetic diversity; and (d) commercial feasibility of progeny. In some embodiments, ensuring diversification may involve limiting the number of crosses that involve a particular parent from the existing germplasm or contain any single pedigree. In some embodiment, promoting commercial feasibility of progeny may involve giving greater preference to selection of maturity groups supported by geographical considerations.

These and other aspects of the disclosure will be further explained below.

### DRAWINGS

The Detailed Description is described with reference to the accompanying figures. The use of the same reference numbers in different instances in the description and the figures may indicate similar or identical items.

FIG. 1 is a diagram of a system and associated methods for selecting recommended crosses with increased probability to meet plant-based product specifications.

FIG. 2A is a diagram of system and associated methods (102) for establishing a phenotype to genotype mapping using machine learning.

FIG. 2B is a diagram of features that may be used to train one embodiment of the system and associated methods for establishing a phenotype to genotype (“PtG”) mapping using machine learning, one embodiment of which being illustrated in FIG. 2A.

FIG. 2C is three graphs illustrating genetic marker density (M) versus overall genetic accuracy for maize, rice and soybean using random marker selection, stride marker selection, and marker thinning approaches.

FIG. 2D illustrates a Manhattan Plot result of a genome-wide association study (GWAS) revealing a SNP associated with a desired phenotypic trait.

FIG. 2E is three graphs illustrating genetic marker density (M) versus overall genetic accuracy for maize, rice and soybean using stride marker selection versus a hybrid marker selection method.

FIG. 2F is a flow chart illustrating a preferred method for hybrid marker selection.

FIG. 2G is an illustration of an improved approach to simulating pairwise combinations of parents available in the germplasm.

FIG. 3 is a diagram illustrating the process of potential changes to one or more of the machine-learning models based on live data collection.

FIG. 4 is a block diagram illustration one potential system within which one or more of the inventive concepts disclosed in the present specification may be implemented.

### DETAILED DESCRIPTION

The present invention now will be described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as methods or devices. The following detailed description is, therefore, not to be taken in a limiting sense.

In the following detailed description of embodiments of the inventive concepts, numerous specific details are set forth in order to provide a more thorough understanding of the inventive concepts. However, it will be apparent to one of ordinary skill in the art that the inventive concepts within the disclosure may be practiced without these specific details. In other instances, certain well-known features may not be described in detail to avoid unnecessarily complicating the instant disclosure.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherently present therein.

Unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

The term “and combinations thereof” as used herein refers to all permutations or combinations of the listed items preceding the term. For example, “A, B, C, and combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. A person of ordinary skill in the art will understand that typically there is no limit on the number of items or terms in any combination, unless otherwise apparent from the context.

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

The use of the terms “at least one” and “one or more” will be understood to include one as well as any quantity more than one, including, but not limited to, each of, 2, 3, 4, 5, 10, 15, 20, 30, 40, 50, 100, and all integers and fractions, if applicable, therebetween. The terms “at least one” and “one or more” may extend up to 100 or 1000 or more, depending on the term to which it is attached; in addition, the quantities of 100/1000 are not to be considered limiting, as higher limits may also produce satisfactory results.

Further, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein qualifiers such as “about,” “approximately,” and “substantially” are intended to signify that the item being qualified is not limited to the exact value specified, but includes some slight variations or deviations therefrom, caused by measuring error, manufacturing tolerances, stress exerted on various parts, wear and tear, and combinations thereof, for example.

As used herein, “components” may be analog or digital components that perform one or more functions. The term “component” may include hardware, such as a processor (e.g., microprocessor), a combination of hardware and software, and/or the like. Software may include one or more computer executable instructions that when executed by one or more components cause the component to perform a specified function. It should be understood that any and all algorithms described herein may be stored on one or more non-transitory memory. Exemplary non-transitory memory may include random access memory, read only memory, flash memory, and/or the like. Such non-transitory memory may be electrically based, optically based, and/or the like.

As used herein, a “mutation” is any change in a nucleic acid sequence. Nonlimiting examples comprise insertions, deletions, duplications, substitutions, inversions, and translocations of any nucleic acid sequence, regardless of how the mutation is brought about and regardless of how or whether the mutation alters the functions or interactions of the nucleic acid. For example and without limitation, a mutation may produce altered enzymatic activity of a ribozyme, altered base pairing between nucleic acids (e.g. RNA interference interactions, DNA-RNA binding, etc.), altered mRNA folding stability, and/or how a nucleic acid interacts with polypeptides (e.g. DNA-transcription factor interactions, RNA-ribosome interactions, gRNA-endonuclease reactions, etc.). A mutation might result in the production of proteins with altered amino acid sequences (e.g. missense mutations, nonsense mutations, frameshift mutations, etc.) and/or the production of proteins with the same amino acid sequence (e.g. silent mutations). Certain synonymous mutations may create no observed change in the plant while others that encode for an identical protein sequence nevertheless result in an altered plant phenotype (e.g. due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements such as tRNAs, ribozymes, ribosome components, and microRNAs.

Methods disclosed herein are not limited to mutations made in the genomic DNA of the plant nucleus. For example, in certain embodiments a mutation is created in the genomic DNA of an organelle (e.g. a plastid and/or a mitochondrion). In certain embodiments, a mutation is created in extrachromosomal nucleic acids (including RNA) of the plant, cell, or organelle of a plant. Nonlimiting examples include creating mutations in supernumerary chromosomes (e.g. B chromosomes), plasmids, and/or vector constructs used to deliver nucleic acids to a plant. It is anticipated that new nucleic acid forms will be developed and yet fall within the scope of the claimed invention when used with the teachings described herein.

Methods disclosed herein are not limited to certain techniques of mutagenesis. Any method of creating a change in a nucleic acid of a plant can be used in conjunction with the disclosed invention, including the use of chemical mutagens (e.g. methanesulfonate, sodium azide, aminopurine, etc.), genome/gene editing techniques (e.g. CRISPR-like technologies, TALENs, zinc finger nucleases, and meganucleases), ionizing radiation (e.g. ultraviolet and/or gamma rays) temperature alterations, long-term seed storage, tissue culture conditions, targeting induced local lesions in a genome, sequence-targeted and/or random recombinases, etc. It is anticipated that new methods of creating a mutation in a nucleic acid of a plant will be developed and yet fall within the scope of the claimed invention when used with the teachings described herein.

Similarly, the embodiments disclosed herein are not limited to certain methods of introducing nucleic acids into a plant and are not limited to certain forms or structures that the introduced nucleic acids take. Any method of transforming a cell of a plant described herein with nucleic acids are also incorporated into the teachings of this innovation, and one of ordinary skill in the art will realize that the use of particle bombardment (e.g. using a gene-gun), Agrobacterium infection and/or infection by other bacterial species capable of transferring DNA into plants (e.g., Ochrobactrum sp., Ensifer sp., Rhizobium sp.), viral infection, and other techniques can be used to deliver nucleic acid sequences into a plant described herein. Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

Methods disclosed herein include conferring desired traits to plants, for example, by mutating sequences of a plant, introducing nucleic acids into plants, using plant breeding techniques and various crossing schemes, etc. These methods are not limited as to certain mechanisms of how the plant exhibits and/or expresses the desired trait. In certain nonlimiting embodiments, the trait is conferred to the plant by introducing a nucleotide sequence (e.g. using plant transformation methods) that encodes production of a certain protein by the plant. In certain nonlimiting embodiments, the desired trait is conferred to a plant by causing a null mutation in the plant's genome (e.g. when the desired trait is reduced expression or no expression of a certain trait). In certain nonlimiting embodiments, the desired trait is conferred to a plant by crossing two plants to create offspring that express the desired trait. It is expected that users of these teachings will employ a broad range of techniques and mechanisms known to bring about the expression of a desired trait in a plant. Thus, as used herein, conferring a desired trait to a plant is meant to include any process that causes a plant to exhibit a desired trait, regardless of the specific techniques employed.

As used herein, “fertilization” and/or “crossing” broadly includes bringing the genomes of gametes together to form zygotes but also broadly may include pollination, syngamy, fecundation and other processes related to sexual reproduction. Typically, a cross and/or fertilization occurs after pollen is transferred from one flower to another, but those of ordinary skill in the art will understand that plant breeders can leverage their understanding of fertilization and the overlapping steps of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of fertilization and/or crossing as used herein when performed in conjunction with these teachings. In certain embodiments, the gametes are not different cell types (i.e. egg vs. sperm), but rather the same type and techniques are used to effect the combination of their genomes into a regenerable cell. Other embodiments of fertilization and/or crossing include circumstances where the gametes originate from the same parent plant, i.e. a “self” or “self-fertilization”. While selfing a plant does not require the transfer pollen from one plant to another, those of skill in the art will recognize that it nevertheless serves as an example of a cross, just as it serves as a type of fertilization. Thus, methods and compositions taught herein are not limited to certain techniques or steps that must be performed to create a plant or an offspring plant of the claimed invention, but rather include broadly any method that is substantially the same and/or results in compositions of the claimed invention.

A “plant” refers to a whole plant, any part thereof, or a cell or tissue culture derived from a plant, comprising any of: whole plants, plant components or organs (e.g., leaves, stems, roots, etc.), plant tissues, seeds, plant cells, protoplasts and/or progeny of the same. A plant cell is a biological cell of a plant, taken from a plant or derived through culture of a cell taken from a plant.

A “population” means a set comprising any number, including one, of individuals, objects, or data from which samples are taken for evaluation, e.g. estimating QTL effects and/or disease tolerance. Most commonly, the terms relate to a breeding population of plants from which members are selected and crossed to produce progeny in a breeding program. A “population of plants” can include the progeny of a single breeding cross or a plurality of breeding crosses and can be either actual plants or plant derived material, or in silico representations of plants. The member of a population need not be identical to the population members selected for use in subsequent cycles of analyses nor does it need to be identical to those population members ultimately selected to obtain a final progeny of plants. Often, a “plant population” is derived from a single biparental cross but can also derive from two or more crosses between the same or different parents. Although a population of plants can comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population in a plant breeding program.

“Crop performance” is used synonymously with “plant performance” and refers to of how well a plant grows under a set of environmental conditions and cultivation practices. Crop performance can be measured by any metric a user associates with a crop's productivity (e.g. yield), appearance and/or robustness (e.g. color, morphology, height, biomass, maturation rate), product quality (e.g. fiber lint percent, fiber quality, seed protein content, seed carbohydrate content, etc.), cost of goods sold (e.g. the cost of creating a seed, plant, or plant product in a commercial, research, or industrial setting) and/or a plant's tolerance to disease (e.g. a response associated with deliberate or spontaneous infection by a pathogen) and/or environmental stress (e.g. drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g. the volume or weight of seed or other plant product measured in liters or grams) or some other metric assigned to some aspect of a plant that can be represented on a scale (e.g. assigning a 1-10 value to a plant based on its disease tolerance).

A “microbe” will be understood to be a microorganism, i.e. a microscopic organism, which can be single celled or multicellular. Microorganisms are very diverse and include all the bacteria, archaea, protozoa, fungi, and algae, especially cells of plant pathogens and/or plant symbionts. Certain animals are also considered microbes, e.g. rotifers. In various embodiments, a microbe can be any of several different microscopic stages of a plant or animal. Microbes also include viruses, viroids, and prions, especially those which are pathogens or symbionts to crop plants.

A “fungus” includes any cell or tissue derived from a fungus, for example whole fungus, fungus components, organs, spores, hyphae, mycelium, and/or progeny of the same. A fungus cell is a biological cell of a fungus, taken from a fungus or derived through culture of a cell taken from a fungus.

A “pest” is any organism that can affect the performance of a plant in an undesirable way. Common pests include microbes, animals (e.g. insects and other herbivores), and/or plants (e.g. weeds). Thus, a “pesticide” is any substance that reduces the survivability and/or reproduction of a pest, e.g. fungicides, bactericides, insecticides, herbicides, and other toxins.

“Tolerance” or improved tolerance in a plant to disease conditions (e.g. growing in the presence of a pest) will be understood to mean an indication that the plant is less affected by the presence of pests and/or disease conditions with respect to yield, survivability and/or other relevant agronomic measures, compared to a less tolerant, more “susceptible” plant. Tolerance is a relative term, indicating that a “tolerant” plant survives and/or performs better in the presence of pests and/or disease conditions compared to other (less tolerant) plants (e.g., a different soybean cultivar) grown in similar circumstances. As used in the art, tolerance is sometimes used interchangeably with “resistance”, although resistance is sometimes used to indicate that a plant appears maximally tolerant to, or unaffected by, the presence of disease conditions. Plant breeders of ordinary skill in the art will appreciate that plant tolerance levels vary widely, often representing a spectrum of more-tolerant or less-tolerant phenotypes, and are thus trained to determine the relative tolerance of different plants, plant lines or plant families and recognize the phenotypic gradations of tolerance.

A plant, or its environment, can be contacted with a wide variety of “agriculture treatment agents.” As used herein, an “agriculture treatment agent”, or “treatment agent”, or “agent” can refer to any exogenously provided compound that can be brought into contact with a plant tissue (e.g. a seed) or its environment that affects a plant's growth, development and/or performance, including agents that affect other organisms in the plant's environment when those effects subsequently alter a plant's performance, growth, and/or development (e.g. an insecticide that kills plant pathogens in the plant's environment, thereby improving the ability of the plant to tolerate the insect's presence). Agriculture treatment agents also include abroad range of chemicals and/or biological substances that are applied to seeds, in which case they are commonly referred to as “seed treatments” and/or seed dressings. Seed treatments are commonly applied as either a dry formulation or a wet slurry or liquid formulation prior to planting and, as used herein, generally include any agriculture treatment agent including growth regulators, micronutrients, nitrogen-fixing microbes, and/or inoculants. Agriculture treatment agents include pesticides (e.g. fungicides, insecticides, bactericides, etc.) hormones (abscisic acids, auxins, cytokinins, gibberellins, etc.) herbicides (e.g. glyphosate, atrazine, 2,4-D, dicamba, etc.), nutrients (e.g. a plant fertilizer), and/or a broad range of biological agents, for example a seed treatment inoculant comprising a microbe that improves crop performance, e.g. by promoting germination and/or root development. In certain embodiments, the agriculture treatment agent acts extracellularly within the plant tissue, such as interacting with receptors on the outer cell surface. In some embodiments, the agriculture treatment agent enters cells within the plant tissue. In certain embodiments, the agriculture treatment agent remains on the surface of the plant and/or the soil near the plant. In certain embodiments, the agriculture treatment agent is contained within a liquid. Such liquids include, but are not limited to, solutions, suspensions, emulsions, and colloidal dispersions. In some embodiments, liquids described herein will be of an aqueous nature. However, in various embodiments, such aqueous liquids that comprise water can also comprise water insoluble components, can comprise an insoluble component that is made soluble in water by addition of a surfactant, or can comprise any combination of soluble components and surfactants. In certain embodiments, the application of the agriculture treatment agent is controlled by encapsulating the agent within a coating, or capsule (e.g. microencapsulation). In certain embodiments, the agriculture treatment agent comprises a nanoparticle and/or the application of the agriculture treatment agent comprises the use of nanotechnology.

In certain embodiments, plants disclosed herein can be modified to exhibit at least one “desired trait”, and/or combinations thereof. The disclosed innovations are not limited to any set of traits that can be considered desirable, but nonlimiting examples include male sterility, herbicide tolerance, pest tolerance, disease tolerance, modified fatty acid metabolism, modified carbohydrate metabolism, modified seed yield, modified seed oil, modified seed protein, modified lodging resistance, modified shattering, modified iron-deficiency chlorosis, modified water use efficiency, and/or combinations thereof. Desired traits can also include traits that are deleterious to plant performance, for example, when a researcher desires that a plant exhibits such a trait in order to study its effects on plant performance.

In certain embodiments, a user can combine the teachings herein with high-density molecular marker profiles spanning substantially the entire soybean genome to estimate the value of selecting certain candidates in a breeding program in a process commonly known as “genomic selection.”

At least in the near term, the answer to meaningful substantive improvement of plant-based products may result from the aggregation of smaller improvements in those products. The disclosed systems and methods can consider billions of data points in millions of pipeline configurations to identify the starting parental plant breeding combinations, predict gene targets, and analyze optimal farm management and environmental conditions to guide eventual placement of improved varieties in the field. This result may more easily be attained by assessing the seeds in germplasm 105 using the machine learning techniques disclosed herein alongside in silico simulation and perhaps also gene editing. In fact, using just machine learning and in silico simulation has already facilitated the rapid identification and development of plant-based (soy) products with ultra-high protein (UHP). The ability to get plant-based products to get to market more efficiently and more quickly may be important in effectively responding to evolving consumer preferences and the needs of growers.

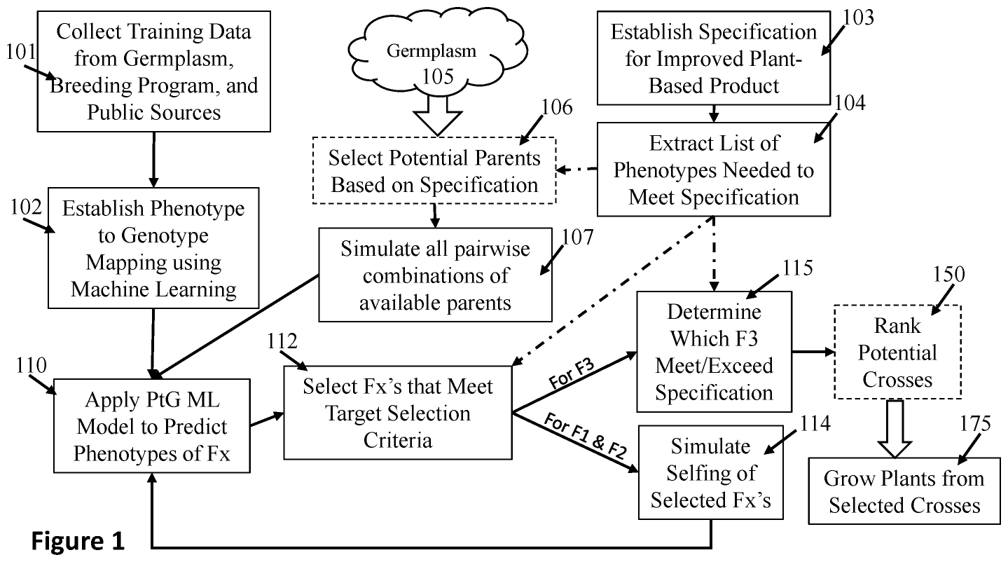

FIG. 1 generally illustrates a system and associated methods for selecting recommended crosses with increased probability to meet plant-based product specifications. As illustrated, the system and method involves collecting training data 101 from germplasm 105, a breeding program and public sources; establishing a phenotype to genotype mapping 102 using machine learning; establishing a specification for an improved plant-based product 103; extracting a list of phenotypes needed to meet the specification 104; selecting potential parents based on the specification 106; simulating all pairwise combinations of the available parents 107; applying the genomic selection model (created by mapping 102) to predict the phenotypes of Fx (i.e., F1, F2 ((selfed F1s), and F3 (selfed F2s)) (110); selecting the Fx's that meet the target selection criteria at the target selection intensity (112) using the list of phenotypes need to meet the specification; for simulated F1 and selfed F2's that meet the selection criteria at the target selection intensity, simulating the “selfing” of each selected Fx (114) or for simulated F3's that meet the selection criteria at the target selection intensity determining how many of them meet or exceed the specification (115); ranking the potential crosses (150); and growing plants from the selected crosses (175).

A. Using Machine Learning to Establish a Phenotype to Genotype Mapping Based on Training Data Collected from the Germplasm, Breeding Program, and Public Sources (101 and 102)

The term “machine learning” generally refers to computer algorithms that may learn from pre-existing data and then make predictions about new data. Thus, machine-learning tools operate by building a model from example training data, which, for example, can be used to model an environment based on that training data and then make decisions or predictions without explicit instructions.

Different machine-learning tools may be used. Deep learning or deep structured learning is a type of machine learning that can use artificial neural networks (e.g., inspired by biological systems) with representation learning. Representation learning is a set of techniques that allows a system to automatically discover representations needed to detect features in future sets of data.

The learning of features is generally thought to be either supervised or unsupervised, although a hybrid of these approaches is also possible.

In “supervised learning,” a “teacher” presents the computer with the desired outputs given a set of example inputs. This is generally thought to involve classification and regression, which can be accomplished using one or more approaches including, but not limited to, decision trees, ensembles (e.g. Random Forest), nearest neighbors algorithm, linear regression, gBLUP (genomic best linear unbiased prediction), lasso (least absolute shrinkage and selection operator), lasso LARS, Ridge regression, Elastic Net, Naive Bayes, Artificial neural networks (ΔNN or NN), logistic regression, perceptron, Relevance vector machine (RVM), and Support vector machine (SVM). Generally, the approach to supervised learning used depends on the data set, among other issues involved in this choice is the amount training data available, the dimensionality and heterogeneity of that data, redundancy in that data, the interrelations between data elements, and the amount of noise present in the output.

In “unsupervised learning,” the computer is left to find any naturally occurring patterns within the training data. This can be accomplished by using one or more approaches including, but not limited to, clustering (i.e., automatically grouping the training examples into categories with similar features), anomaly detection, principal component analysis (i.e., automatically identifying features that are most useful for discriminating between different training examples and then discarding the rest), self-organizing feature maps, and latent variable models. Clustering methods include hierarchical clustering, k-means, mixture models (i.e., a probabilistic model that represents the presence of subpopulations within an overall population), DBSCAN (density-based spatial clustering of applications with noise), expectation-maximization, BIRCH, and CURE.

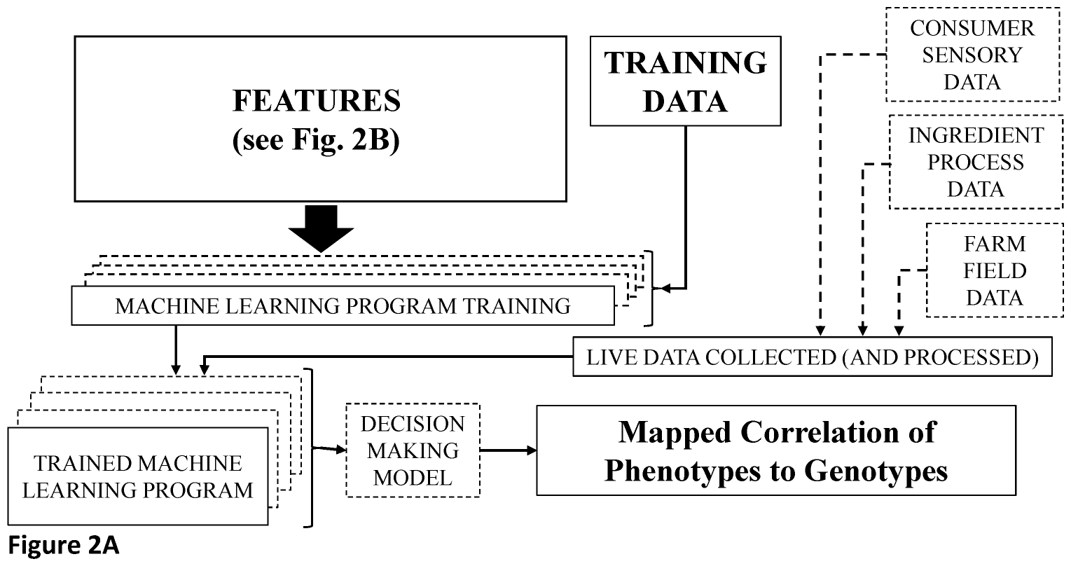

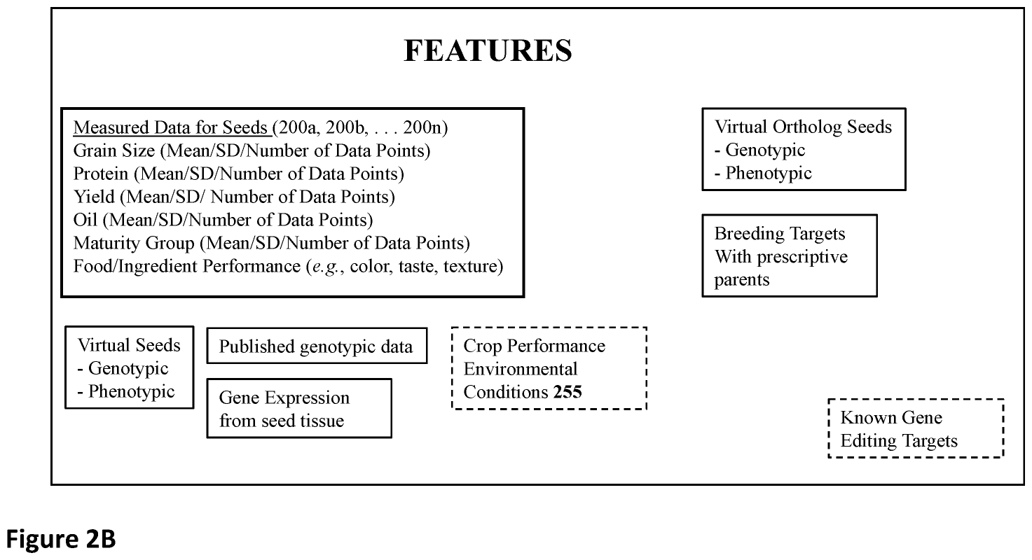

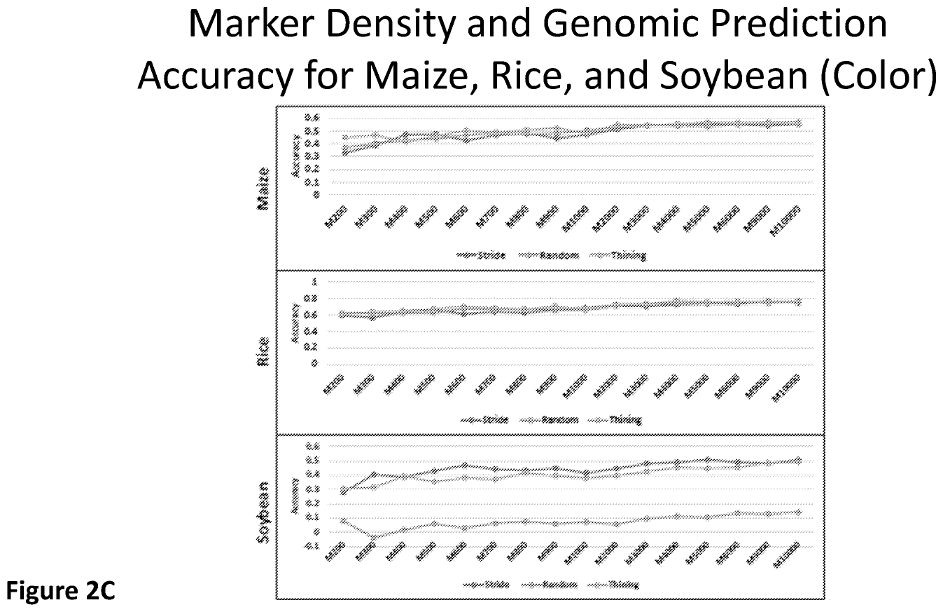

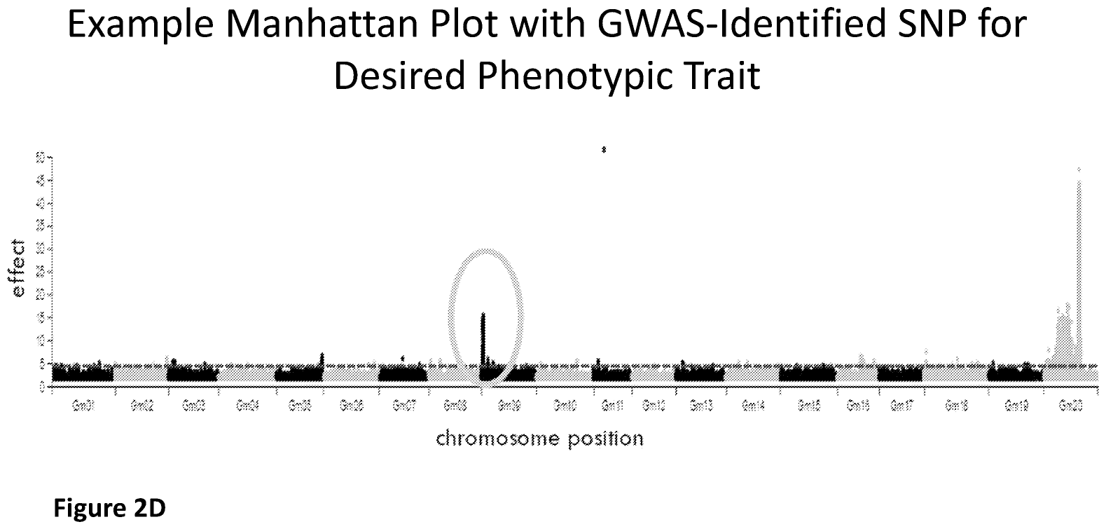

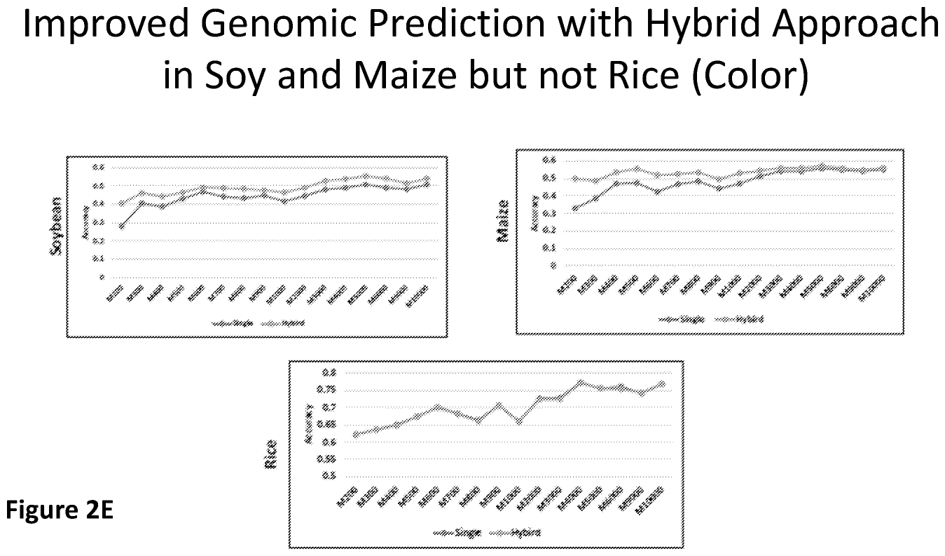

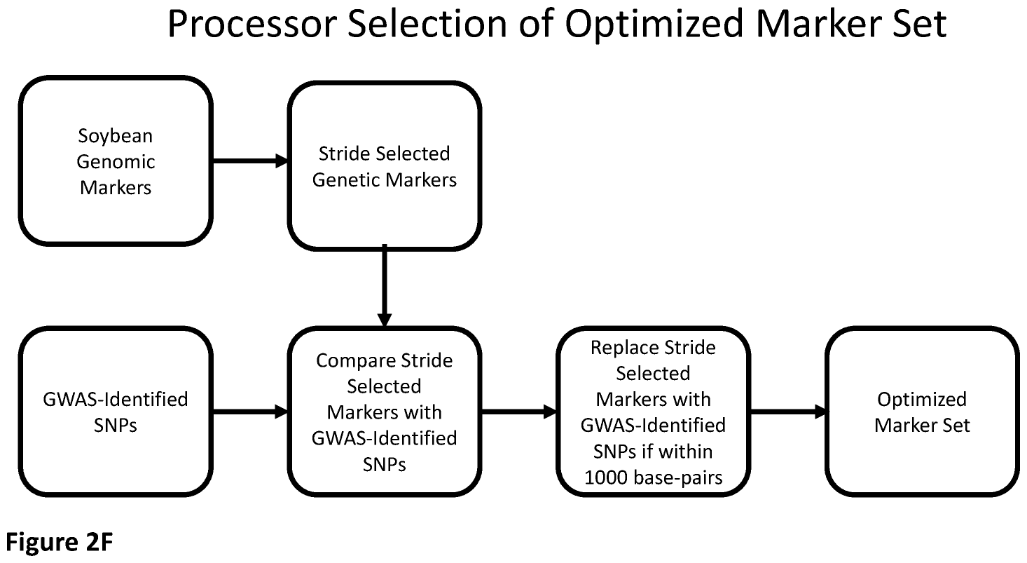

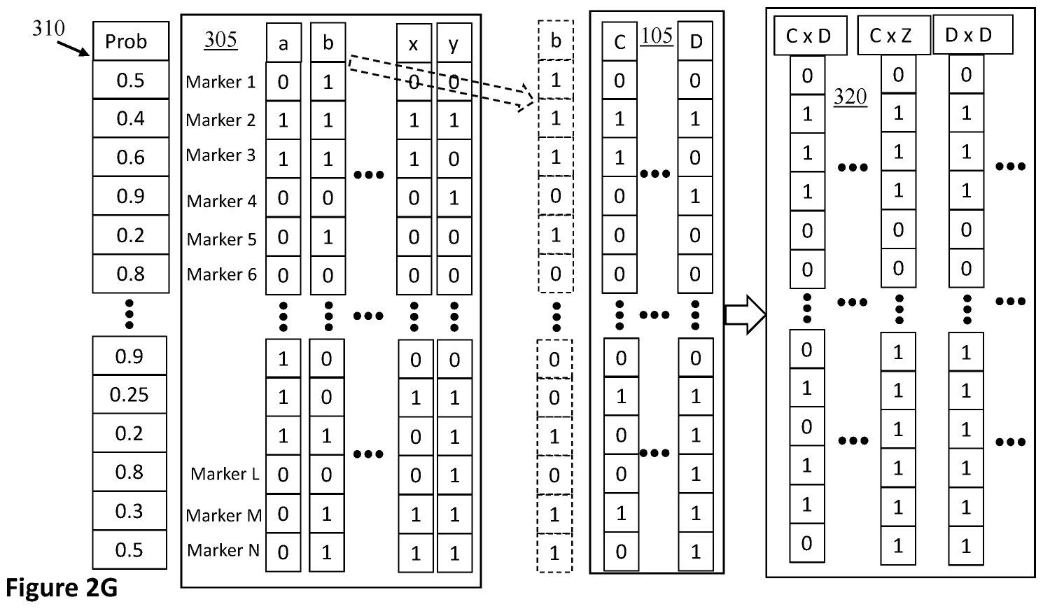

As collectively illustrated by FIGS. 2A and 2B, one or more of the foregoing supervised and unsupervised machine learning approaches may be used by the present system and methods in parallel or seriatim using the same training data or subsets thereof. Where subsets are used the scope of any such subset may be selected for use with the particularly selected training data within that subset with reference to the pluses and minuses of one or more of the particular approaches to machine learning. Where multiple machine learning approaches are used in parallel (i.e., stacked) a decision-making model is preferably introduced to mediate between the probability assessments provided by the multiple machine learning models toward providing a single list of recommended actions (e.g., desirable plant crosses, gene editing targets, crop management techniques).

Training machine learning models requires the selection of features and collection of data associated with relevant features in order to appropriately train the machine learning model. As illustrated across FIGS. 2A and 2B, the present disclosure identifies various categories of data that the inventors believe may play a substantive role in training useful models. As noted in block 101, this data may be collected from the germplasm 105, from a breeding program, and/or from public sources. For instance, one public source that may be utilized in training a model for use with soybeans (Glycine max and/or G. Soja) is SoyBase (which was accessed on Dec. 23, 2022 via the internet at soybase.org), Another potential public source of data may be publicly available academic research on soybeans. As for data collected from these public sources, as well as from the germplasm 105 and/or breeding program, as illustrated in FIG. 2B, each of those collected data may be saved to a seed object (or seed vector) 200a, 200b, to 200n that describes each unique seed contained within the germplasm 105. The seed object 200 is preferably identified by one or more of its germplasm ID, its parentage, genotypic, phenotypic, or other genetic data. Seed object 200 may be virtual in the sense that it may contain nothing more than the germplasm ID, parentage and basic genetic data.

A “virtual” seed object 200 may also include genomic forecasted probabilities for the seed such as protein content, yield, oil content, and maturity group, all of which may be represented as their mean values and may have an associated standard deviation. These less fulsome objects/vectors still play a substantive role in simulation and ML (machine learning) decision making. However, additional data collected may be added to these seed object/vectors that improve the ability of the models to evaluate and make recommendations with respect to subsequent decisions as to mapping phenotypes to genotypes, as well as to potentially other aspects of the system (e.g., future crosses, recombination, seed advancement, and seed deployment).

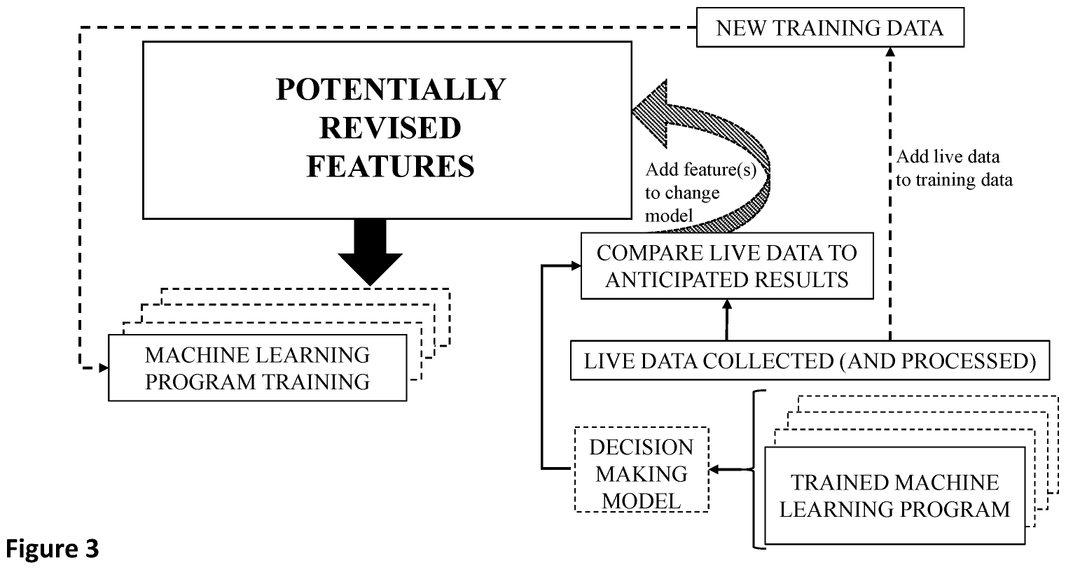

As illustrated by FIG. 3, physical testing data may be collected and may be further processed based on directions from the machine learning system. Processing may be performed on the directly observed physical data (e.g., genotype, phenotype, genetic sequencing (partial or WGS), ingredient processing data, and consumer sensory data) or on one or more derivative data sets (e.g., GWAS or TWAS) based on the observed physical data. The directly observed data may be collected during indoor growing exercises, in field testing, and commercialization and/or from the results of such indoor growing exercises, field testing, and commercialization by obtaining tissue samples from the various steps in the process. For example, tissue samples may be obtained from seeds generated during indoor growing exercises (e.g., speed breeding) which may be subjected genotyping, sequencing (partial or WGS), and/or predictive phenotyping. In another example, tissue samples taken from seeds resulting from the growth of an F4 generation may be subjected to both food testing protocols as well as genotyping/sequencing/predictive phenotyping, whereas seeds resulting from commercialization may only be subjected to food testing protocols. Thus, during and following one or more these events, information may be gathered and subsequently recorded to a seed object 200 associated with a particular seed.

As there will be a variable number of observations across each of the seed object/vector 200 associated with the germplasm 105 as the system 100 continues to operate, the equal variance assumption that underlies linear ML models will not be met. Consequently, operations in the present system may better lend themselves to neural network analysis. Moreover, because neural networks allow the system to capture relationships even where certain outlier values may be “too high” or “too low,” these NNs may provide additional advantages to the system over linear and other models with respect to real-time agricultural decision making.

The data saved to seed objects 200a-n may also include measured data for a seed (really a population of seeds sharing a common pedigree). As illustrated in FIG. 2B, for soybeans this measured seed data may include protein, yield, oil, Maturity Group, and food testing protocol data for each instance that seed is grown. The protein and oil data may be further measured and recorded as to type of protein/oil. Crop Performance environmental conditions data 255 for each instance the seed has been grown and observed may also preferably be associated with this collected data. Crop Performance environmental conditions data 255 may be collected from indoor growing exercises or in-field growth. Crop Performance environmental conditions data 255 may include location (e.g., latitude/longitude for in-field growth), soil health (e.g., its microbiome and Nitrogen content), climate data (e.g., rain/watering frequency and amount, sunlight length and intensity, air temperature, soil temperature), and applied crop management practices (e.g., planting depth, plant spacing, planting date, irrigation, fertilizer use). As noted above, the field data 255 collected with respect to any particular growing event may not produce instructive data with respect to all of these variables (e.g., the location of an indoor growing event) or even where all of the variables could have been collected, the data may not have been recorded, entered into the dataset, or removed from the dataset for various reasons. In this regard, the models selected for use with the overall dataset contemplate the potential absence of data points from the overall dataset.

As illustrated in FIG. 2B, the seed records 200a-n may include the number of actual data points collected with respect to each separate data type, as well as the mean and standard deviation for that data, and may further include various correlations, such as the correlation of observed protein to observed yield. It should be understood by those of ordinary skill in the art having the present disclosure before them that correlations may be calculated and included in a seed object data record 200, such as correlations, if any, between protein and oil, protein and maturity group, protein and food testing data, yield and oil, yield and maturity group, yield and food testing data, oil and maturity group, oil and food testing data, and maturity group and food testing data. It may further be possible using the collected data to identify opportunities to use growing data from one or more prior growing season in predicting future performance of the seed. Thus, for example, the probabilities with respect to future protein and yield of a seed, are significantly improved when combining genomic prediction with prior year field data (e.g. use the measured results of Phase 1 field testing to predict Phase 2 results).

The genotypic data may include, but is not limited to, ATAC-Seq, gene annotation, gene expression, genes essential development and maintenance, GO (Gene Ontology) Terms, GWAS (genome wide association study) data, known QTL (quantitative trait locus) data, known eQTL (expression quantitative trait locus), expression data, co-expression data, metabolites data, promoters, RNA-sequencing data (preferably collected at R4 and R5), structural variant (SV) data, transcriptome data, TWAS (transcriptome-wide association study) data, and WGS (whole genome sequencing) data. The matched transcriptome and WGS data may comprise the entirety (or nearly the entirety) of the DNA sequence of an organism's genome. It is further envisioned that a collection of genotypes, some of which may be “haplotypes” at loci that are clustered together on the same chromosome, as well as collections of genotypes from across a single chromosome, and/or collections of genotypes corresponding to loci distributed on different chromosomes may be measured, saved, and used in one or more of the various models operating within the present system.

ATAC-Seq is a technique to assess genome-wide chromatin accessibility. Gene expression links to tissues and times when a particular gene is active allowing for a direct link of gene level changes to phenotypic changes, at scale. Gene Ontology is a representation of detectable observations in genes and relationships between those observations, which allows scientists to publish specific observations about genes opening up literature as a source of training data. GWAS data is a method of studying associations between a genome-wide set of single-nucleotide polymorphisms (SNPs) and a desired phenotypic traits, such as increased protein content. QTL is the location within a genome that correlates with a variation in a quantitative phenotype of the organism.

As illustrated in FIG. 3, it is contemplated that some of the data may be collected and fed back into the model continuously. Other data, such as expression data, while it is high value corelative data particularly with respect to protein content in soybeans, is expensive to generate. Assuming a scenario where genotype data for 5,000 samples is approximately $135,000, expression data for just four replicates, 2 tissues would be approximately $9,000,000. In such instances it would be ideal to find a proxy for such data. Continuing with the example of expression data, expression values can be predicted using already collected expression data correlated to other genotypic data. Using predicted expression data allows the system to dramatically increase sample numbers and the power of the machine learning model. In particular, by using the predicted expression for more than 6,300 genes across 1800+ soybean lines along with protein measurements for those same 1800+ soybean lines as training data for a random forest regression machine-learning model, high predictive accuracy has been obtained.

The phenotypic data may include various desirable and undesirable traits associated with a particular plant. For example, with respect to plants used in making plant-based protein product phenotypic data may include the protein content in seeds of the plant (measured both in the field using NIR and in a wet lab), the density of other nutrients in the seeds of the plant, the oil content in seeds of the plant, the oleic acid content in seeds of the plant, the fiber content in the seeds of the plant, the oligosaccharides content (e.g., raffinose and stachyose) in seeds of the plant, the saponins/isoflavones/PUFA content in the seeds of the plant, the content of other off-flavor contributing chemicals (e.g., Hexanal and Hexanol) in the seeds of the plant, the moisture content in seeds of the plant (water holding capacity), plant height, the yield history for the plant, the maturity group (MG) of the plant, and environmental stress resistance of the plant.

It would also be desirable to integrate into the “design” of new plant breeds, food science data for the resulting plant-based end-products, including consumer sensory panel data (e.g., taste (bitterness, richness, saltiness, sourness, umami), texture, firmness, color) and supply chain insights. For instance, water usage, energy usage, and overall cost of producing products based on a particular plant breed design should be fed back into the machine learning model as features to allow for additional improvements in this regard.

### B. Establishing a Specification for an Improved Plant-Based Product and Extracting a List of Phenotypes Need to Meet the Specification (103 and 104)

For example, if an improved soy-protein based hamburger is desired, the process begins by setting product specifications, which could include increased protein content, increased water holding capacity, improved flavor, and decreased total oil. The specification could also require that ingredient processing be as energy-efficient as possible to meet growing consumer preferences. In another example, desired specifications for soybean-based white beverage (e.g., soy milk) could include increased protein content, increased solubility, improved flavor, improved color, and a differentiated saturated fat profile. In yet another example, a soybean-based egg replacement, the specification may include increased emulsion/foaming, increased gelation, increased water holding capacity, and decreased total oil. Based on each particular specification, the necessary traits for the ultimately desired commercial soybean for that specification would be established. Then, the work of selecting plants to cross in order to breed a plant that achieves those desired traits in a commercial soybean plant begins.

As recognized in the prior art, selective breeding to achieve desired traits in plant products take years. In the method generally illustrated by FIG. 1, the desired traits (e.g., maximized protein content, minimized oligosaccharides, increased water holding capacity), may be assessed against the genetic information and phenotypes of plants within an available germplasm to predict and potentially rank the most efficient paths (e.g., quickest, most cost-effective, most environmentally friendly, and combinations thereof) that have the highest probabilities of achieving the desired specification. In many cases, some traits will be easier to integrate through gene editing than breeding. In other cases, gene targets believed to result in the desired traits may be yet unknown, too difficult to edit/modify successfully, provide insufficient improvement of the desired trait, or may otherwise prove undesirable. In some cases, a combination of breeding crosses and genetic editing will provide the most efficient path to the desired end product specification. In still other cases, breeding, genetic editing, planting location and crop management techniques will provide the most efficient path with the highest probability of producing an end product that meets (or exceeds) the specification.

In particular, once a specification is established for the improved plant-based product (103), the list of plant traits (e.g., protein content, oil content, water holding capacity, yield, decreased/muted chemical expression) that are believed necessary to meet (or exceed) that specification are extracted from the specification (104). Additional specifications may be established around other potentially desirable (or even undesirable) crop/plant performance characteristics (many potential characteristics are discussed above).

Once the list of phenotypes has been extracted and a phenotype-to-genotype mapping established, it would now be possible to establish a predictive crossing plan. At its most basic, a predictive crossing plan is based on the calculated probability of the progeny of two parents meeting product thresholds and maximizing genetic value with respect to one or more traits (e.g., protein content). In a preferred embodiment of the present method, the crossing plan is established by simulating all of the pairwise combinations of available parents (107), applying the genomic selection model (i.e., phenotype-to-genotype mapping); selecting the simulated progeny in each generation (i.e., F1, selfed F2 and F3) that meet or exceed the genomic selection criteria (established in element 104); and then determine (115) how many F3 meet or exceed the plant-based product specification and ranking the remaining crosses (150).

### C. Improved Genetic Simulation to Support Plant Development Crossing Plan (107)

The genetic simulation may be performed using a rapid recombination simulator. In a preferred embodiment, the rapid recombination simulator models the distance between the start of chromosome and each crossover point and one crossover to the next, using a statistical stochastic model, such as a gamma or Poisson distribution. The genetic map is measured in Morgans, which are a measure of probability. As such, any distribution could be used, including, but not limited to bionomial/Bernoulli, uniform, normal, and specialized gamma distributions (e.g., Erlang). However, at present, it is believed that gamma or Poisson distributions would be the most appropriate for this application.

In a preferred embodiment, the model may be established beginning with a publicly-available genetic map. (For soybeans, for example, such a genetic map for one or more soybean varieties is publicly available via soybase.org). While an entire genetic map of a plant genome may be used for the simulation, in a preferred embodiment, a reduced set of genetic markers may be used to reduce the number of chromosomes/crossover points to be simulated. Using fewer genetic markers in simulation is desirable (so long as genetic accuracy remains acceptable) because, among other things, simulating fewer markers increases processing efficiency (i.e., decreased processor usage, increased speed).

As discussed below, the size of the set of markers simulated may be reduced in a thoughtful fashion while sufficiently maintaining the genomic predictive capabilities of the system and method. There are at least three approaches to reducing the set of markers: random marker selection, stride marker selection and marker thinning.

I. Random marker selection: a random selection of n markers from an overall genomic map for the desired plant.

II and III. Stride marker selection and marker thinning: In stride marker selection, every n^{th} genomic marker is retained, where n is equal to a representative set of markers divided by target set of markers. This selection approach results in more markers in the high recombination areas. In marker thinning, the markers are evenly distributed across the genome.

FIG. 2C is three graphs each illustrating genetic marker density (M) versus overall genetic accuracy for maize, rice and soybean, respectively, when using random marker selection, stride marker selection, or marker thinning approaches to reducing the number of genetic markers for each type of plant. Among other things, the data in FIG. 2C illustrates an increase in accuracy as marker density increases for both stride marker selection and thinning marker selection methods for soybeans. It also shows that both stride marker selection and thinning marker selection methods are much more accurate than the random selection method for soybeans. For corn/maize, the data of FIG. 2C further illustrates a potential increase in accuracy for the thinning selection method as compared to the stride-selection or random selection methods for a lower number of markers, but at higher marker density, there is no significant increase in accuracy between the thinning and stride selection methods compared to the random method for corn. FIG. 2C also shows an increase in accuracy for all markers in rice, with neither the stride selection or thinning selection approaches resulting in a significantly higher prediction accuracy than the random marker selection approach.

Focusing on the results for soybeans, the data shows generally with respect to the stride market selection method that as marker density increases genomic prediction accuracy increases, with the highest prediction accuracy being when n=5000 and n=10000. FIG. 2C also shows that a high level of genomic prediction accuracy is possible with fewer markers for soybean, maize, and rice. Again focusing on soybeans, FIG. 2C shows that when n=600 the genomic prediction accuracy for stride marker selection method was approximately the same as when n=3000. In fact, with respect to soybeans, the data of FIG. 2C indicates that for all n values both the stride and thinning marker selection methods were more accurate than a random marker selection method. Still, as would be further recognized by one skilled in the art of genomics having the present results and discussion before them, while the thinning marker selection method yielded significantly more accurate results than a random selection method, it would be far less desirable to create an even distribution of genetic markers when assessing the potential progeny of self-pollenating plants because recombination rates of such plants vary across their genomes. Thus, for self-pollenating plants, a stride selection method would be preferred over the thinning marker method because stride marker selection should produce higher genomic prediction accuracy results than a random method and should result in a subset of genetic markers that are clustered around higher recombination areas. (Interestingly, the data of FIG. 2C indicates that while rice is a self-pollenating plant, the stride selection method did not yield greater genomic prediction in rice.) For cross pollenating plants, such as maize/corn, recombination rates are distributed more evenly across their genome. Accordingly, a thinning select method may be preferred for maize/corn.

IV. Stride-GWAS hybrid selection: In addition to random marker selection, stride marker selection and marker thinning approaches to marker set reduction, it may be possible to create a hybrid approach to reduce the size of the market set used in simulation by combining, for instance, the stride marker selection method with a genome-wide association study (GWAS). GWAS is a method to study associations between single-nucleotide polymorphism (SNPs) and desired phenotypic traits. With respect to soybeans, such phenotypic traits may include, but are not limited to, protein content, oil content, and yield. In GWAS, desired phenotypic traits are compared to SNPs and analyzed on a Manhattan plot, as illustrated by FIG. 2D. By using GWAS, significant (p≤0.0005) SNPs for desired phenotypic traits can be determined.

Thus, as illustrated in FIG. 2F, one preferred approach to selecting an optimized marker set may begin by selecting genetic markers from a more fulsome list genomic markers (e.g., a publicly-available map of a soybean) based on a stride marker selection method. The selected markers may then be compared markers identified by GWAS (e.g., for soybeans desirable markers would be associated, for example, with ultra-high protein content, quality protein, high oil content, and high yield) where a GWAS-identified SNPs is within preferably approximately 1000 base-pairs of a marker selected via the stride marker selection technique, the stride-selected marker is replaced with the GWAS-identified marker. As shown in FIG. 2E (a graph illustrating marker density versus genetic accuracy results for both hybrid and stride selection techniques for soybean, maize, and rice), the hybrid Stride-GWAS selection model provides at least a modest improvement in accuracy when used with maize (at a lower marker density) and soybeans (across the board) for marker densities ranging between 200 and 10,000 markers.

In an alternative approach, a thinning-hybrid selection approach can be utilized for select pollenating plants. Researchers anticipate that a thinning-GWAS hybrid approach would utilize a substantially similar methodology as a stride-GWAS hybrid approach. A thinning-GWAS hybrid approach would be more effective in cross-pollenated plants where recombination rates are more likely to be evenly distributed across the genome.

The rapid recombination simulator is illustrated, in part, by FIG. 2G. In particular, the rapid recombination simulator involves pre-computed crossover “hash maps” 305a-y. Each of these crossover vectors, 305a-y, is a binary encoded representation of recombinations, each representing a hypothetical crossover outcome between any two arbitrary parents.

Each hash map 305a-y has N markers. Based on the foregoing discussion, N may equal the total number of markers mapped for the plant being simulated (e.g., soybean, rice or corn) or it may equal a smaller number of markers based on the use of a reduced market set (established in the manner discussed immediately above).

In a preferred embodiment, the crossover vectors, 305a-y are computed by taking representations of a variety of plant vectors (having the same N markers) from the germplasm 105
and with a probability of 0.5, flip “1” (i.e., True) to “0” (i.e.. False) and vice versa flip “0” to 1. So, assuming the following example crossover vector (shortened for the sake of explanation) is sampled: 1010, where the probability is 1.0, the example crossover vector would be flipped to 0101. However, because the probability is 0.5, there is only a 50% chance for each bit that any bit will be flipped. In other words, the following results would be possible using the shortened example where the probability is 0.5:0000, 0001, 0010, 0011, 0100, 0101, 0110, 0111, 1000, 1001, 1010, 1011, 1100, 1101, 1110, 1111. These illustrative examples are for explanation only, as only one result will be generated for each bit in each crossover vector, e.g. 0111. As illustrated in FIG. 2G, it may be desirable to apply a different probability to each marker by creating an array 310 of such probabilities, which may be based on data gathered from a breeding program, preferably associated with germplasm 105.

As illustrated in FIG. 2G, during simulation, a pre-computed crossover vector, for example 305b, is randomly sampled/selected from among crossover vectors 305a-y. And, then the selected crossover vector 305 is used to simulate meiosis with recombination, taking parents from germplasm 105, for example parent C and parent D (where C may equal D), for each loci in the crossover vector, e.g., 305b, where “1” (i.e. True) the corresponding bit value is taken from the first haplotype (e.g., parent C), where it is “0” (i.e., False) the corresponding bit value is taken from the second haplotype (e.g., parent D). The result of this process using parents C and D and crossover vector 305b is illustrated by “C×D” in table 320. So, in this example, the crossover vector at “Marker 1” is “1,” Hap 1 at “Marker 1” is 0, so “Marker 1” in “C×D” is “0” because the simulation takes the value from Marker 1 of the first haplotype (i.e., Parent C) when the value of the crossover vector at the corresponding loci is “1.”

Significant efficiencies arise as a result of applying this simulation applying these pre-computed crossover vectors 305a-y to parents from the germplasm 105. In fact, it may be desirable to use pre-existing libraries in Numpy (“Numerical Python”) to leverage crossover vectors (305a-305y) and process the still very large number of binary entries using the simulation rules (described in the preceding paragraph) to simulate probable progeny for all combinations of the (eligible) parents in germplasm 105. The inventors compared the amount of processing time needed to simulate 20 million progeny using this novel approach versus AlphaSimR (publicly-available via https://CRAN.R-project.org/package=AlphaSimR. See, R Chris Gaynor, Gregor Gorjanc, John M Hickey, AlphaSimR: an R package for breeding program simulations, G3 Genes|Genomes|Genetics, Volume 11, Issue 2, February 2021, jkaa017, https://doi.org/10.1093/g3journal/jkaa017. The difference in time to needed to process 20 million progeny on AlphaSimR versus the novel approach disclosed in the present disclosure with similar genomic accuracy was slightly greater than 45 minutes (i.e., approximately 5 minutes versus nearly 55 minutes).

As further illustrated in FIG. 1, the parents available for the simulation may be pre-limited before beginning the simulation with reference to the desired specifications (106). For example, if the desired plant-based product specifications calls for ultra-high protein (UHP), the parents selected from the germplasm 105 may be limited to those parents that exhibit UHP. Another example may be whether a potential parent produces a high content of oleic oil. By limited the available parents for simulation, the process may be even more efficient.

Using the genomic model created in element 102, in element 110, phenotypes are predicted for each of the results (e.g., “C×D”, “C×Z”, “D×D” in table 320) resulting from the simulation in element 107. Then, in element 112, the simulated progeny that meets (or exceeds) the target selection criteria are selected. For instance, the system and method may be searching for soybean progeny that are expected to achieve a yield of greater than/equal to 60 bushels per acre with a white flake maximized to 63%. The selection in element 112 may be further limited in some embodiments to progeny that not only meets/exceeds the target selection criteria, but further meets a target selection intensity (e.g., advance only the top 10%).

As illustrated in FIG. 1, for each progeny selected for advancement in element 112 for the simulated F1 and F2 generations, a subsequent simulation is conducted again using the rapid recombination simulator (described above in association with FIG. 2G) except that each selected progeny is simulated as combining with itself (i.e., selfing). So, for example, let's say that progeny “A×Y” (see FIG. 2G, element 320) was advanced forward by element 112, the next generation would be simulated by the rapid recombination simulator using “A×Y” as both the first and the second haplotype. As illustrated in FIG. 1, phenotypes are predicted (element 110) for each of the results of the self'ed simulations. Then, in element 112, the simulated progeny that meets (or exceeds) the target selection criteria are selected. The selection in element 112 may be further limited in some embodiments to progeny that not only meets/exceeds the target selection criteria, but further meets a target selection intensity (e.g., advance only the top 10%). It is also contemplated that even where the F1 and/or F2 generation was not limited by target selection intensity in element 112 that the F2 or F3 could still be so limited.

Once the F3 generation has been simulated (element 114), the GS model to predict phenotypes applied (element 110), and the F3's that meet the target selection criteria (and potentially also the target selection intensity) selected, the system and method determines which of the resulting F3 meet and/or exceed the specification (created by elements 103 and 104). A list of F3 candidates (and the crosses behind such candidates) is advanced forward by element 115.

In a preferred embodiment, the potential crossing list advanced forward by element 115 may be ranked (element 150). The ranking may be designed to promote one or more of the following goals: (1) maximizing one or more desired phenotypic results (e.g., white flake protein content in soybean or yellow pea; high oleic oil content in soybean; yield; low raffinose and stachyose fixed in both parents; tolerance to environmental stressors); (2) ensuring diversification across maturity subgroups; (3) ensuring (and perhaps even promoting) genetic diversity in the germplasm 105; and (4) commercial feasibility of progeny. Constraints used in the ranking (element 150) may be particular to certain types of plants (e.g., high oleic oil soybeans, ultra-high protein (UHP) soybeans). With respect to ensuring diversification, ranking may limit the number of crosses, for example involving one particular parent from the germplasm or containing any single pedigree (e.g., no UHP-Quality Protein×UHP-Quality Protein). With respect to commercial feasibility, ranking may skew selection toward maturity groups supported by geographical considerations (e.g., maturity range of available farmland; distance of available farmland to crushing facilities).

Once the crosses are ranked, plants may be grown based on the selected crosses list. Such growing may be conducted using speed breeding, which is likely to be conducted within an indoor facility that provides controlled growing conditions (e.g., temperature, daily photoperiod, humidity) year around without unintentional stressors (e.g., insects, drought). In speed breeding, the daily photoperiod is longer resulting expedited growth in the plants. Tissue samples may be collected from the plants in the speed breeding program. These tissue samples may be subjected to a variety of physical tests, such as genotyping, sequencing, and predictive phenotyping. In this regard, where a primary specification needed for the resulting plant-based product is increased protein, one type of physical data gathered from the plants may comprise certain NIR data. This NIR data may be correlated to predict protein content in soybeans. The NIR data may be obtained by applying NIR light directly to soybeans, soybean pods, or even soy plants, but most preferably the NIR light is applied directly to the beans. Where other product specifications are sought, other physical testing may be done, as may be appropriate, given the specification and the particular stage in the pipeline (e.g., F1, F2, F3, F4, Commercialization).

Some amount of genotype data may also be collected between generations. The collection of this genomic data allows for assessment of the model and better future predictions. Where genomic data of a line significantly deviates from the genomic predictions of the model (especially if that deviation suggests negative future performance), that line may not be further advanced through breeding. Data gathered at every stage may be included as part of the training data toward improving and/or even re-establishing one or more of the machine learning models for future use within the system. As seeds progress through the pipeline, the information regarding the seed and the ability to calculate its probability of successfully meeting the product specification increases. Conversely, as the seeds progress through the pipeline the number of alternative paths decreases.

### EXAMPLE APPLICATIONS

Example 1: Soy, specifically soy protein concentrate (SPC) is the number one protein ingredient used in plant-based meat applications. SPC has a protein content of approximately 65%. Currently, SPC is primarily made by processing of defatted soy flour (approximately 47% protein content) produced from soybeans with an average protein content of approximately 36%. The processing required to increase the protein content is costly, water-intensive, and energy-intensive. It is believed that an ultra-high protein (UHP) soybean could make this process less expensive, less water-intensive, and more energy-efficient. By leveraging the soybean plant's genetic diversity, its protein content may be increased to a sufficiently high-level (at least 49%) that it would effectively disintermediate one or more processing steps necessary to arrive at the protein level suitable for plant-based meat applications. As the protein content of the soybeans in the field is driven toward 65%, the less waste and processing that would be required to produce Soy Protein Concentrate.

Example 2: It is anticipated that better soybean genetics can be found in a germplasm which includes, among other varieties, the wild ancestor of the present day commercial soybean, Glycine soja (previously G. ussuriensis) or created using lessons from that broader germplasm and/or orthologs that will (a) facilitate other easier, cheaper, more environmentally friendly production of soy-based ingredients, potentially alleviating supply constraints; (b) allow for the production of completely new ingredients (e.g., de-flavored, high-water holding capacity soybeans for enhanced flavor and texture in final plant-based meat products; healthy oils (due to higher oleic acid); stable gelation); (c) new food products; and/or (d) improved end user satisfaction (e.g., better taste, texture, color)).

Example 3: Soybean meal is an ideal protein source for swine, poultry, and fish due to its availability, cost, high protein content, and balanced amino acid profile. In fact, currently over 90% of the soybeans produced in the United States are fed to animals. However, its use has been restricted because—like many plant proteins—soybean meal has a high concentration of antinutritional compounds (ANCs), including oligosaccharides such as raffinose and stachyose that can have a negative effect on protein digestibility, leading to low energy values, poor metabolism, and excessive secretion impacting water quality in aquaculture systems. Apart from antinutritional factors, the steady decline in protein content of soy—an unintended consequence of breeding primarily for yield and other agronomic traits—has rendered soy meal a continually less valuable feed ingredient. Through machine learning it is anticipated that the expression of oligosaccharides such as raffinose and stachyose can be significantly decreased.

Example 4: The yellow pea is another significant source of plant-protein. Currently, pea protein concentrate (PPC) has a protein content of approximately 50-52% and pea protein isolate (PPI) a protein content of about 85%. The flavor and color of PPC is not preferred by consumers. While PPI has better flavor, the cost of process is much higher. Using machine learning to understand and optimize the diversity of yellow pea parents in a germplasm and subsequently optimize and prioritize the crosses most likely to succeed, it is believed that the protein content of the yellow pea in the field can be significantly increased much like the protein content of unprocessed soybeans is being increased. It is also believed that machine learning models will help identify the gene(s) that result in the undesirable flavor and color of the yellow pea and provide gene editing actions to mute/lessen the undesirable flavor and color to provide greater consumer interest in yellow-pea based food ingredients. This will (a) facilitate other easier, cheaper, more environmentally friendly production of yellow-pea-based ingredients, alleviating plant-protein supply constraints; (b) allow for the production of completely new ingredients (e.g., de-flavored, high-water holding capacity yellow peas for enhanced flavor and texture in final plant-based meat products); (c) new food products; and/or (d) improved end user satisfaction (e.g., better taste, texture, color).

### Computing Environment to Support Machine Learning

It should also be noted that the machine learning models, data collection, various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, and so on).

Aspects of the methods and systems described herein, such as the logic or machine learning models, may be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

Aspects of the methods and systems disclosed herein may be embodied and/or executed by the logic of the processes described herein, which may also be embodied in the form of software instructions and/or firmware that may be executed on any appropriate hardware. For example, logic embodied in the form of software instructions and/or firmware may be executed on a dedicated system or systems, on a personal computer system, on a distributed processing computer system, and/or the like. In some embodiments, logic may be implemented in a stand-alone environment operating on a single computer system and/or logic may be implemented in a networked environment such as a distributed system using multiple computers and/or processors, for example.

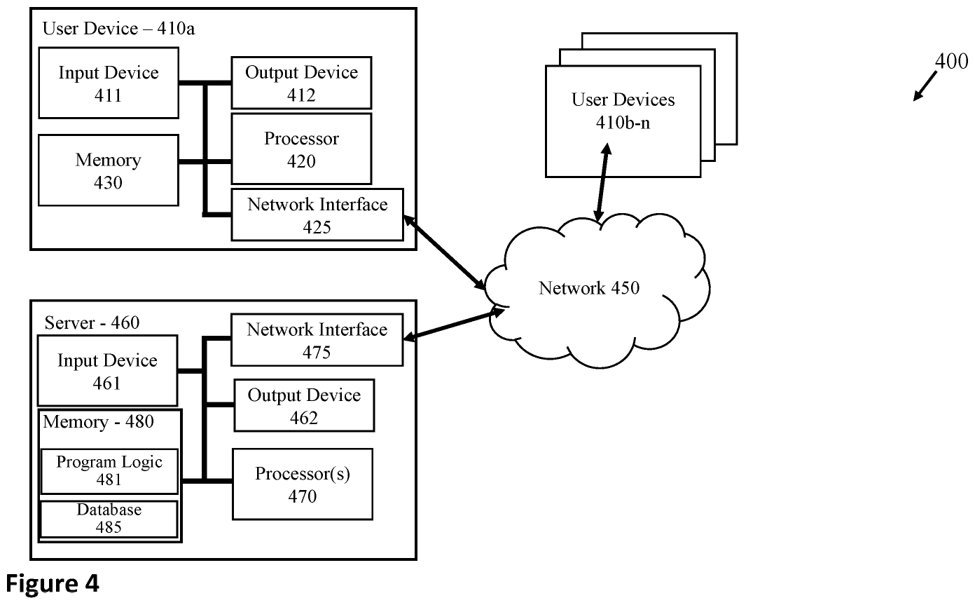

Aspects of the methods and systems described herein may also be implemented on an illustrative system 400, depicted in association with FIG. 4. In particular, system 400 may comprise a user devices 410a-n, server 460, and network 450.

The user device 410 of the system 400 may include various components including, but not limited to, one or more input devices 411, one or more output devices 412, one or more processors 420, a network interface device 425 capable of interfacing with the network 450, one or more non-transitory memories 430 storing processor executable code and/or software application(s), for example including, a web browser capable of accessing a website and/or communicating information and/or data over the network, and/or the like. The memory 430 may also store an application (not shown) that, when executed by the processor 420 causes the user device 410 to provide the functionality of the various systems and methods described the present specification, as would be understood by those of ordinary skill in the art having the present specification, drawings, and claims before them.

The input device 411 may be capable of receiving information input from the user and/or processor 420, and transmitting such information to other components of the user device 410 and/or the network 450. The input device 411 may include, but are not limited to, implementation as a keyboard, touchscreen, mouse, trackball, microphone, remote control, and combinations thereof, for example.

The output device 412 may be capable of outputting information in a form perceivable by the user and/or processor 420. For example, implementations of the output device 412 may include, but are not limited to, a computer monitor, a screen, a touchscreen, an audio speaker, a website, and combinations thereof, for example. It is to be understood that in some exemplary embodiments, the input device 411 and the output device 412 may be implemented as a single device, such as, for example, a computer touchscreen. It is to be further understood that as used herein the term “user” is not limited to a human being, and may comprise, a computer, a server, a website, a processor, a network interface, a user terminal, and combinations thereof, for example.

The server 460 of the system 400 may include various components including, but not limited to, one or more input devices 461, one or more output devices 462, one or more processors 470, a network interface device 475 capable of interfacing with the network 450, and one or more non-transitory memories 480 for storing data structures/tables (including those of database 485) that may be used by the system 400 and particularly server 460 to perform the functions and procedures set forth herein. The memory 480 may also store an application/program store 481 that, when executed by the processor 470 causes the server 460 to provide the functionality of the systems and methods disclosed in the present application.

As shown in FIG. 4, the server 460 may include a single processor or multiple processors working together or independently to execute the program logic 481 stored in the memory 480 as described herein. It is to be understood, that in certain embodiments using more than one processor 470, the processors 470 may be located remotely from one another, located in the same location, or comprising a unitary multi-core processor. The processors 470 may be capable of reading and/or executing processor executable code and/or capable of creating, manipulating, retrieving, altering, and/or storing data structures and data tables (including those of database 485) into the memory 480.

Exemplary embodiments of the processor 470 may be include, but are not limited to, a digital signal processor (DSP), a central processing unit (CPU), a field programmable gate array (FPGA), a microprocessor, a multi-core processor, combinations, thereof, and/or the like, for example. The processor 470 may be capable of communicating with the memory 480 via a path (e.g., data bus). The processor 470 may be capable of communicating with the input device 461 and/or the output device 462.

The input device 461 of the server 460 may be capable of receiving information input from the user and/or processor 470, and transmitting such information to other components of the server 460 and/or the network 450. The input device 461 may include, but are not limited to, implementation as a keyboard, touchscreen, mouse, trackball, microphone, remote control, and/or the like and combinations thereof, for example. The input device 461 may be located in the same physical location as the processor 470, or located remotely and/or partially or completely network-based.

The output device 462 of the server 460 may be capable of outputting information in a form perceivable by the user and/or processor 470. For example, implementations of the output device 462 may include, but are not limited to, a computer monitor, a screen, a touchscreen, an audio speaker, a website, a computer, and/or the like and combinations thereof, for example. The output device 462 may be located with the processor 470, or located remotely and/or partially or completely network-based.

The memory 480 stores applications or program logic 481 as well as data structures (including those of database 485) that may be used by the system 400 and particularly server 460. The memory 480 may be implemented as a conventional non-transitory memory, such as for example, random access memory (RAM), CD-ROM, a hard drive, a solid state drive, a flash drive, a memory card, a DVD-ROM, a disk, an optical drive, combinations thereof, and/or the like, for example. In some embodiments, the memory 480 may be located in the same physical location as the server 460, and/or one or more memory 480 may be located remotely from the server 460. For example, the memory 480 may be located remotely from the server 460 and communicate with the processor 470 via the network 450. Additionally, when more than one memory 480 is used, a first memory 480a may be located in the same physical location as the processor 470, and additional memory 480n may be located in a location physically remote from the processor 470. Additionally, the memory 480 may be implemented as a “cloud” non-transitory computer readable storage memory (i.e., one or more memory 480 may be partially or completely based on or accessed using the network 450).

Each element of the server 460 may be partially or completely network-based or cloud-based, and may or may not be located in a single physical location. As used herein, the terms “network-based,” “cloud-based,” and any variations thereof, are intended to include the provision of configurable computational resources on demand via interfacing with a computer and/or computer network, with software and/or data at least partially located on a computer and/or computer network. In other words, the server 460 may or may not be located in single physical location. Additionally, multiple servers 460 may or may not necessarily be located in a single physical location.

Database 485 may comprise one or more data structures and/or data tables stored on non-transitory computer readable storage memory 480 accessible by the processor 470 of the server 460. The database 485 can be a relational database or a non-relational database. Examples of such databases include, but are not limited to: DB2®, Microsoft® Access, Microsoft® SQL Server, Oracle®, mySQL, PostgreSQL, MongoDB, Apache Cassandra, and the like. It should be understood that these examples have been provided for the purposes of illustration only and should not be construed as limiting the presently disclosed inventive concepts. The database 485 can be centralized or distributed across multiple systems.

The teachings herein are not limited to certain plant species, and it is envisioned that they can be modified to be useful for monocots, dicots, and/or substantially any crop and/or valuable plant type, including plants that can reproduce by self-fertilization and/or cross fertilization, hybrids, inbreds, varieties, and/or cultivars thereof. Some of example plant species include, soybeans (Glycine max), peas (Pisum sativum and other members of the Fabaceae like Cjanus and Vigna species), chickpeas (Cicer arietinum), peanuts (Arachis hypogaea), lentils (Lens culinaris or Lens esculenta), lupins (various Lupinus species), mesquite (various Proopis species), clover (various Trifolium species), carob (Ceratonia siliqua), tamarind, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), tomato (Solanum lycopersicum), lettuce (Lactuca sativa), safflower (Carthamus tinctorius), wheat (Triticum aestivum), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), eucalyptus (Eucalyptus spp.), oats (Avena sativa), barley (Hordeum vulgare), flax (Linum usitatissimum), Buckwheat (Fagopyrum esculentum) vegetables, ornamentals, and conifers.

While particular embodiments of the present invention have been shown and described, it should be noted that changes and modifications may be made without departing from the presently disclosed inventive concepts in its broader aspects and, therefore, the aim in the appended claims is to cover all such changes and modifications as fall within the true spirit and scope of this invention.

What is claimed is:

1. A method for selecting recommended crosses from a population of plants in a germplasm with an increased probability of meeting a plant-based product specification, comprising:
(a) collecting into a database, with a processor, plant data for the population of plants in the germplasm, such plant data comprising at least labelled parentage information that includes genetic and phenotype information;
(b) training, with the processor, a machine learning model mapping phenotypes to genotype based on the data collected into the database;
(c) extracting, via the processor, a target selection list including one or more phenotypes needed to meet the plant-based product specification;
(d) simulating, via the processor, pairwise combinations of one or more available parents using rapid recombination simulation;
(e) applying, via the processor, the phenotype-to-genotype mapping to predict one or more phenotypes for each simulated pairwise combination;
(f) selecting, via the processor, the simulated pairwise combinations that at least meets phonetic criteria on the target selection list;
(g) simulating, via the processor, selfed combinations of each selected simulated pairwise combinations using rapid recombination simulation;
(h) repeating (e) through (g) until an F3 generation has been simulated; and
(i) creating a predictive crossing list of simulated F3 progeny that meets the product specification.

2. The method according to claim 1 further comprising ranking potential crosses on the predictive crossing list to promote one or more goals selected from the group comprising: (a) maximizing one or more desired phenotypic results; (b) ensuring diversification across maturity subgroups; (c) ensuring genetic diversity; and (d) commercial feasibility of progeny.

3. The method according to claim 2 wherein ensuring diversification further comprises limiting the number of crosses that involve a particular parent from the existing germplasm or contain any single pedigree.

4. The method according to claim 2 wherein promoting commercial feasibility of progeny may comprise giving greater preference to selection of maturity groups supported by geographical considerations.

5. A system for selecting recommended crosses from a population of plants in a germplasm with an increased probability of meeting a plant-based product specification, comprising:
(a) a database containing plant data for the population of plants in the germplasm, such plant data comprising at least labelled parentage information that includes genetic and phenotype information;
(b) a processor operably connected to the database that (1) trains a machine learning model mapping phenotypes to genotype based on the data collected into the database, (2) extracts a target selection list including one or more phenotypes needed to meet the plant-based product specification, (3) simulates pairwise combinations of one or more available parents using rapid recombination simulation, (4) applies the phenotype-to-genotype mapping to predict one or more phenotypes for each simulated pairwise combination, (5) selects the simulated pairwise combinations that at least meets phonetic criteria on the target selection list, (6) simulates selfed combinations of each selected simulated pairwise combinations using rapid recombination simulation, repeats (4) through (6) until an F3 generation has been simulated; and (7) creates a predictive crossing list of simulated F3 progeny that meets the product specification.

6. The system according to claim 5 wherein the processor further ranks potential crosses on the predictive crossing list to promote one or more goals selected from the group comprising: (a) maximizing one or more desired phenotypic results; (b) ensuring diversification across maturity subgroups; (c) ensuring genetic diversity; and (d) commercial feasibility of progeny.

7. The system according to claim 6 wherein ensuring diversification further comprises limiting the number of crosses that involve a particular parent from the existing germplasm or contain any single pedigree.

8. The system according to claim 6 wherein promoting commercial feasibility of progeny may comprise giving greater preference to selection of maturity groups supported by geographical considerations.
